(19) United States
(12) Patent Application Publication
(10) Pub. No.: US 2022/0152267 A1
(43) Pub. Date: May 19, 2022

(54) WOUND DRESSING

(71) Applicant: CYMMETRIK ENTERPRISE CO., LTD., Taipei (TW)

(72) Inventors: You-Syuan Gao, Taipei City (TW); Shan-Jen Kuo, Taipei City (TW); Jen-Chieh Wei, Taipei City (TW); Che-Ling Chang, Taipei City (TW)

(21) Appl. No.: 16/952,026

(22) Filed: Nov. 18, 2020

(51) Int. Cl. A61L 15/22; A61F 13/02

(52) U.S. Cl. CPC A61L 15/22; A61F 2013/00187; A61F 13/0203

(57) ABSTRACT

A wound dressing includes a substrate, an insulating layer, at least one ion sustainable-released body, and at least one electrode. The insulating layer is disposed on the substrate, with the at least one ion sustainable-released body being uniformly disposed at the insulating layer. The ion sustainable-released body includes ions. The electrode is disposed on the insulating layer, and the electrode and the ions are functioned as an electron donor and an electron acceptor respectively.

### BACKGROUND OF THE INVENTION

### 1. Field of the Invention

The present invention relates to a wound dressing, and more particularly, to a self-generating electrical wound dressing.

### 2. Description of the Prior Art

Wound dressing is a most simple treatment for reducing infection or stimulating cell repair. Recently, various designs of wound dressings have been developed and used in wound-care, in order to hasten the wound healing process. For example, an external power supply may be additionally used to promote wound healing by increasing the mechanism of cell growth. However, the external supplement of currents or voltages is commonly high, which may cause discomfort to patients and aggravate the pain at the wound. On the other hand, additional bactericidal agents such as silver electrode may also be used on some wound dressings to avoid wound inflammation. However, the additional bactericidal agents may have serious cytotoxicity, leading possible harms to human cells. Thus, there is still a crucial need to provide new design of wound dressing so as to meet the therapeutic product requirements.

### SUMMARY OF THE INVENTION

It is one of the primary objectives of the present invention to provide a wound dressing, in which the ion releasing rate of the anode and/or the cathode is highly controllably, so as to effectively improve the wound healing under excellent safety and lower cytotoxicity for cells.

To achieve the purpose described above, one embodiment of the present invention provides a wound dressing including a substrate, an insulating layer, at least one ion sustainable-released body, and at least one electrode. The insulating layer is disposed on the substrate, with the at least one ion sustainable-released body being disposed at the insulating layer. The ion sustainable-released body includes ions. The electrode is disposed on the insulating layer, and the electrode and the ions are functioned as an electron donor and an electron acceptor respectively.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 is a schematic diagram illustrating a wound dressing according to a first embodiment of the present invention.

FIG. 2 is a schematic diagram illustrating a wound dressing according to a second embodiment of the present invention.

FIG. 3 is a schematic diagram illustrating an enlarge view of an insulating layer of a wound dressing according to another embodiment of the present invention.

FIG. 4 is a schematic diagram illustrating a cell repairing data of a control sample.

FIG. 5 is a schematic diagram illustrating a cell repairing data of a wound dressing according to the second embodiment of the present invention.

FIG. 6 is a schematic diagram illustrating a cell repairing data of a comparison sample.

### DETAILED DESCRIPTION

For better understanding of the presented disclosure, preferred embodiments will be described in detail. The preferred embodiments of the present invention are illustrated in the accompanying drawings with numbered elements.

In the present invention, the formation of a first feature over or on a second feature in the description may include embodiments in which the first and second features are formed in direct contact, and may also include embodiments in which additional features may be formed between the first and second features, such that the first and second features may not be in direct contact. In addition, the present invention may repeat reference numerals and/or letters in the various examples. This repetition is for the purpose of simplicity and clarity and does not in itself dictate a relationship between the various embodiments and/or configurations discussed. Furthermore, spatially relative terms, such as “beneath,” “below,” “lower,” “over,” “above,” “upper” and the like, may be used herein for ease of description to describe one element or feature's relationship to another element(s) or feature(s) as illustrated in the figures. The spatially relative terms are intended to encompass different orientations of the device in use or operation in addition to the orientation depicted in the figures. For example, if the device in the figures is turned over, elements described as “below” and/or “beneath” other elements or features would then be oriented “above” and/or “over” the other elements or features. The apparatus may be otherwise oriented (rotated 90 degrees or at other orientations) and the spatially relative descriptors used herein may likewise be interpreted accordingly.

It is understood that, although the terms first, second, third, etc. may be used herein to describe various elements, components, regions, layers and/or sections, these elements, components, regions, layers and/or sections should not be limited by these terms. These terms may be only used to distinguish one element, component, region, layer and/or section from another region, layer and/or section. Terms such as “first,” “second,” and other numerical terms when used herein do not imply a sequence or order unless clearly indicated by the context. Thus, a first element, component, region, layer and/or section discussed below could be termed a second element, component, region, layer and/or section without departing from the teachings of the embodiments.

As disclosed herein, the term “about” or “substantial” generally means within 20%, preferably within 10%, and more preferably within 5%, 3%, 2%, 1%, or 0.5% of a given value or range. Unless otherwise expressly specified, all of the numerical ranges, amounts, values and percentages disclosed herein should be understood as modified in all instances by the term “about” or “substantial”. Accordingly, unless indicated to the contrary, the numerical parameters set forth in the present invention and attached claims are approximations that can vary as desired.

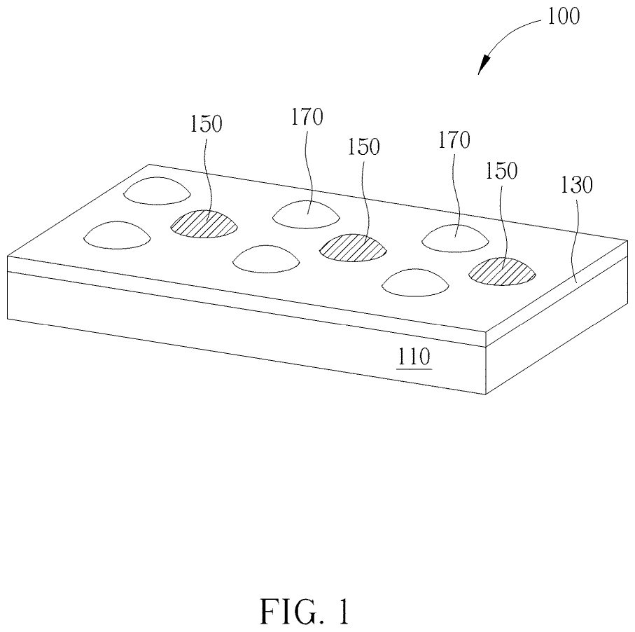

Please refer to FIG. 1, which illustrate a wound dressing 100 according to the first embodiment of the present invention, the wound dressing 100 includes a substrate 110, an insulating layer 130 and an electrode layer sequentially stacked from bottom to top. The substrate 110 is used to maintain the position of the wound dressing 100, and which may include a rigid material or a flexible material having good adhesion, for example the rigid material may include bioceramics, silicate glass, borate glass or the like, and the flexible material may include polytetrafluoroethylene, silicone resin, polyurethane foam, elastomer, synthetic sponge, natural sponge, bio cellulose, non-woven, elastic bandage, breathable waterproof PU film, TPU film, silk, keratin, cellulose fiber, rayon, modified polyacrylonitrile fiber, polyamide film, polyester film, polyolefin film, polyvinyl alcohol film or the like, but not limited thereto. In a preferably embodiment, the substrate 110 may include a silicone material, alginate, fish skin, collagen, chitosan, or a common pressure glue such as PSA glue or an artificial skin, but is not limited thereto. The substrate 110 may optionally include a monolayer structure as shown in FIG. 1, or include a multilayer structure, wherein the multilayer structure may further include an adhesive layer (not shown in the drawings) for further attaching the substrate 110 to the films (including the insulating layer 130 and the electrode layer) disposed thereon, and/or a glue layer (not shown in the drawings) for improving healing, both disposed over the aforementioned rigid material or the flexible material. In one embodiment, the adhesive layer for example includes hypoallergenic sealant, gecko sealants, mussel sealants, waterproof sealants or the like, and the glue layer for example includes poly-acrylic acid, poly-alkyl acrylate, poly-methacrylic acid, poly-methyl methacrylate, poly-2-hydroxyethyl methacrylate, poly-glycidyl methacrylate or the like, but is not limited thereto.

The insulating layer 130 is disposed on the substrate 110, at least covering a portion of a surface of the substrate 110. In FIG. 1, although the insulating layer 130 is exemplified by being disposed on the entire surface of the substrate 110, the practical disposition of the insulating layer 130 is not limited thereto. People skilled in the art should fully realize that the insulating layer may also be disposed on partial surface of the substrate 110 according to various therapeutic product requirements. In one embodiment, the insulating layer 130 for example includes an insulating polymer material for isolating the electrode layer disposed thereon, such as polymer film, rubber, polyurethane material, polyethylene, polyethylene terephthalate, thermoplastic polyurethane (TPU), thermoplastic polyester elastomer (TPEE), biocompatible resin or a combination thereof, but not limited thereto.

The electrode layer is disposed on the insulating layer 130, and which further includes a plurality of anode electrodes 150 and a plurality of cathode electrodes 170, as shown in FIG. 1. In one embodiment, the anode electrodes 150 and the cathode electrodes 170 are both formed on the surface of the insulating layer 130 for example through a printing process, wherein each of the anode electrodes 150 and each of the cathode electrodes 170 may respectively include an electrode for example including potassium (K), sodium (Na), calcium (Ca), magnesium (Mg), aluminum (Al), carbon (C), zinc (Zn), chromium (Cr), iron (Fe), tin (Sn), lead (Pb), hydrogen (H), copper (Cu), mercury (Hg), silver (Ag), platinum (Pt) or gold (Au), with the anode electrodes 150 being configured as a reducing agent in an oxidation-reduction (redox) system or a catalyze facilitating a reducing reaction to include a relative more active element, and with the cathode electrodes 170 being configured as an oxidant agent in the redox system or a catalyze facilitating an oxidation reaction to include a relative less active element, but not limited thereto. Preferably, the anode electrodes 150 and the cathode electrodes 170 have a difference of the standard potentials there between, for example being about 0.05 to 0.5 volts (V), but not limited thereto. In a preferably embodiment, the anode electrodes 150 include a zinc electrode, and the cathode electrodes 170 include a silver oxide (Ago), but is not limited thereto. In this way, the anode electrodes 150 and the cathode electrodes 170 may together form an electrochemical cell, and then micro-currents may be self-generated from the electrode layer when an ion delivery substance (not shown in the drawings) exists. In one embodiment, the ion delivery substance may include physiological saline, wound exudate, or sterile liquid medicine such as medical alcohol, medical hydrogen peroxide, iodophor, red syrup or purple syrup, but not limited thereto.

It is noted that, the anode electrodes 150 and the cathode electrodes 170 are separately disposed from each other without directly in contact with each other, and a distance between each of the anode electrodes 150 and each of the cathode electrodes 170 is about 0.5 to 2 millimeters (mm), so as to generate a low level of micro-currents for example being about 0.1 to 30 microamperes (μA), preferably being 1-20 μA, but not limited thereto. Precisely speaking, while the ion delivery substance exists, the anode electrodes 150 and the cathode electrodes 170 may indirectly contact with each other to conduct the redox chemical reaction, with the anode electrodes 150 to function like an electron donor, performing an oxidation reaction to release electron and anode ions, and with the cathode electrodes 170 to function like an electron acceptor, performing a reduction reaction to receive electron, thereby generating micro-currents via an ion exchanging process to improve the wound healing. Generally, the redox chemical reaction between the anode electrodes 150 and the cathode electrodes 170 will be conducted at a certain consumption rate for a period of time until the anode electrodes 150 and the cathode electrodes 170 are completely exhausted.

People in the art should fully understand that the number, the patterns, the size, and the distribution of the anode electrodes 150 and the cathode electrodes 170 as shown in FIG. 1 are only for example, and which may be further adjustable according to the practical therapeutic purposes. Preferably, the number of the anode electrodes 150 and the cathode electrodes 170 are able to generate at least one micro-current along a horizontal direction (not shown in the drawings, for example the x-direction) after the wound dressing 100 is activated. Then, the anode ions generated from the anode electrodes 150 and the cathode ions generated from the cathode electrodes 170 are gradually released therefrom respectively under the action of potential, so that, the voltage between the anode electrodes 150 and the cathode electrodes 170 is gradually decreased accordingly, till being decreased to zero. In some situation, the materials of the anode electrodes 150 and the cathode electrodes 170 may provide further therapeutic effects, so as to maintain the treatment after the anode electrodes 150 and the cathode electrodes 170 are exhausted. For example, in the preferable embodiment, the silver of the cathode electrodes 170 further provides additional antibacterial effect, which may improve the wound healing thereby.

Through these arrangements, the wound dressing 100 according to the first embodiment of the present invention is provided. In the present embodiment, the electrode layer of the wound dressing 100 is allowable to self-generate micro-currents while existing the ion delivery substance, and the wound dressing 100 may be applied on various damaged, inflamed, or infected biological tissues, for improving the healing process of those biological tissues.

People skilled in the arts should easily realize the design of the wound dressing in the present invention is not limited to the aforementioned embodiment, and may further include other examples or variations. The following description will detail the different embodiments of the wound dressing in the present invention. To simplify the description, the following description will detail the dissimilarities among the different embodiments and the identical features will not be redundantly described. In order to compare the differences between the embodiments easily, the identical components in each of the following embodiments are marked with identical symbols.

Another embodiment of the present invention further provides a wound dressing in which the ion releasing rate may be highly controlled, so as to further improving the healing function thereof. It is noteworthy that the ion releasing rate of the anode electrodes 150 and the cathode electrodes 170 in the aforementioned embodiment are mainly determined according to the rate of the redox chemical reaction, and which may not be artificially controlled by any reaction parameter, thus that, the level of the micro-currents self-generated on the wound dressing 100 thereby may not as expected sometime. In addition, the excessive ion releasing rate of the anode electrodes 150 and/or the cathode electrodes 170 may further lead to serious biological toxicity, thereby resulting in poor therapeutic function of the wound dressing 100. Also, the micro-currents self-generated on the wound dressing 100 are primary concentrated on the surface of the insulating layer 130, between the anode electrodes 150 and the cathode electrodes 170 in the horizontal direction, and the uneven current distribution of the wound dressing 100 makes the wound dressing 100 of the aforementioned embodiment less effective and less practical.

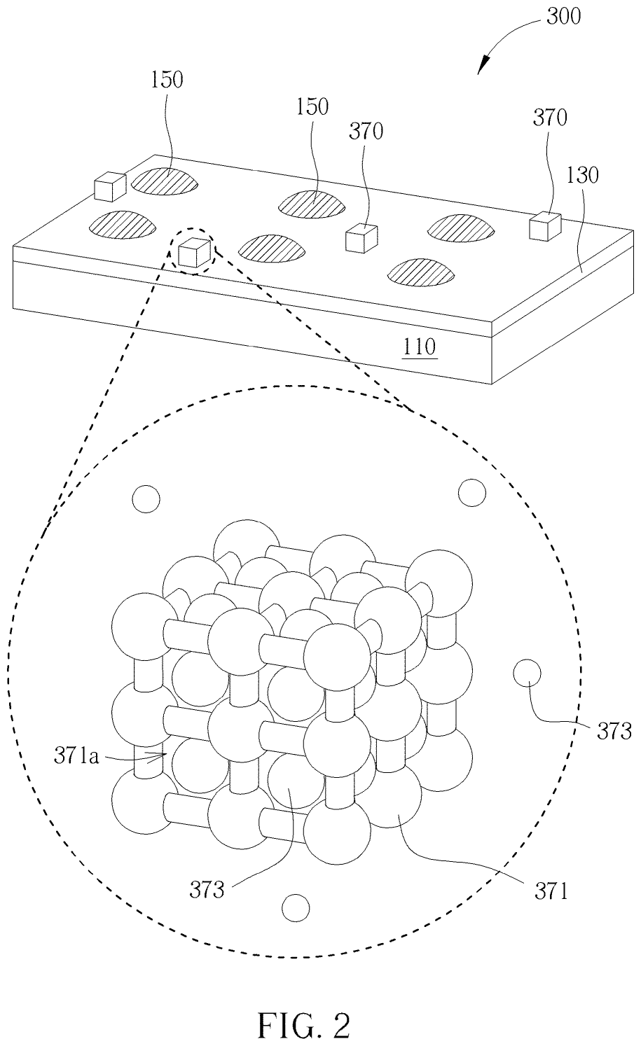

Please refer to FIG. 2, which illustrate a wound dressing 300 according to the second embodiment of the present invention. The formal structures of the present embodiment are substantially similar to those in the aforementioned first embodiment, and the similarity between the present embodiment and the aforementioned embodiment will not be redundantly described hereinafter. The difference between the present embodiment and the aforementioned embodiment is in that the electrode layer of the present embodiment only includes a single electrode such as the anode electrodes 150, with another electrode such as the cathode electrode being omitted, and at least one ion sustainable-released body 370 is further disposed at the insulating layer 130 for highly controlling the cathode ions released therefrom.

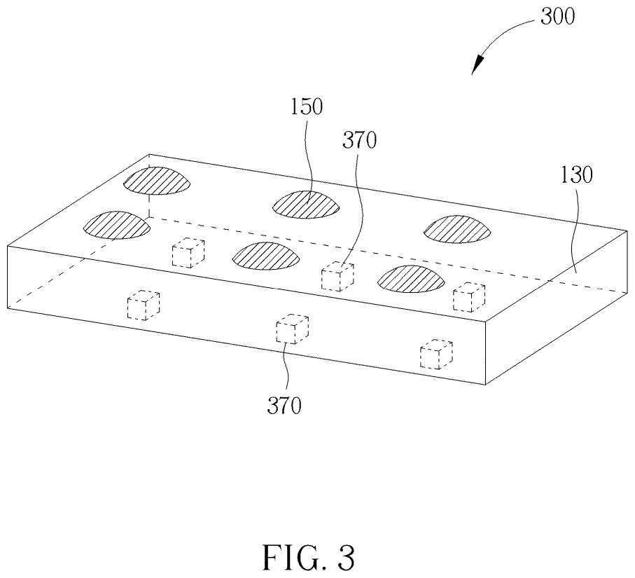

In the present embodiment, a plurality of the ion sustainable-released bodies 370 is disposed at the insulating layer 130. Preferably, the ion sustainable-released bodies 370 may be formed through a printing process, so that, each of the ion sustainable-released bodies 370 may be uniformly distributed on the insulating layer 130 as shown in FIG. 2. The ion sustainable-released bodies 370 may be optionally disposed on a portion of the insulating layer 130, or disposed on entire surface of the insulating layer 130, based on practical requirements. Precisely speaking, each of the ion sustainable-released bodies 370 includes a carrier 371, and a plurality of ions 373 being covered by the carrier 371, wherein the ion sustainable-released bodies 370 are distributed with respect to the insulating layer 130 with a weight ratio of about 0.01% to 10%, preferably about 0.5% to 2%, but not limited thereto. People skilled in the art should fully realize that the practical disposing number or the disposing positions of the ion sustainable-released body 370 is not limited to be shown in FIG. 2, and which may include further variation based on practical product requirements. For example, in another embodiment, the ion sustainable-released bodies 370 may also be uniformly disposed within a portion of the film or within the entire film of the insulating layer 130, as shown in FIG. 3, wherein the ion sustainable-released bodies 370 are also distributed with respect to the insulating layer 130 with a weight ratio of about 0.01% to 10%, preferably about 0.5% to 2%, but not limited thereto.

In other words, the ions 373 are embedded within the carrier 371, with the coverage of the carrier 371 to interfere with the naturally release of ions 373, and then, the releasing rate of ions 373 is slow down and controlled by the coverage of the carrier 371. It is noted that, the releasing rate of ions 373 may be highly controlled by the material selection of the carrier 371, as well as the weight ratio between the carrier 371 and the ions 373. In one embodiment, the carrier 371 for example includes any possible material having a plurality of micro-channels 371a, such as a semiconductor material like silicon or aluminosilicate, an insulating material like mineral, clay, or filter, or a combination thereof, so that, the ions 373 may be sustainable-released from the micro-channels 371a to the insulating layer 130. Then, the releasing rate of ions 373 may be controlled by the size of the micro-channels 371a on the carrier 371, with the ions 373 being faster released through the micro-channels in a relative greater size, and with the ions 373 being slower released through micro-channels in a relative smaller size. In one embodiment, the size of the micro-channels 371a may be in atomic size, for example being ranged from about 1 angstrom (Å) to 10 nanometers (nm), but not limited thereto. People in the art should fully realize that the detailed size of the micro-channels 371a may be further adjusted according to the predicted ion releasing rate or the selected material of the carrier 371, and which is not limited to be the aforementioned size. On the other hand, the ions 373 are distributed with respect to the carrier 371 with a weight ratio of about 1% to 5%, preferably about 2.5%, but not limited thereto. In some embodiments, the releasing rate of ions 373 may be further slow down by additionally disposing a coating layer (not shown in the drawings) outside the ion sustainable-released bodies 370.

Through these arrangements, as the releasing rate of ions 373 from the ion sustainable-released bodies 370 are effectively controlled, the reaction rate of the redox chemical reaction between the anode electrodes 150 and the ion sustainable-released bodies 370 is also effective controlled (slowdown) thereby. In one embodiment, the anode electrodes 150 and the ions 373 of the ion sustainable-released bodies 370 are respectively performed like an electron donor and an electron acceptor in an redox system, wherein the material of the electron donor and the electron acceptor may include potassium, sodium, calcium, magnesium, aluminum, carbon, zinc, chromium, iron, tin, lead, hydrogen, copper, mercury, silver, platinum or gold, but not limited thereto. In the present embodiment, the anode electrodes 150 is configured as a reducing agent or a catalyze facilitating reduction reaction in the redox system to include a relative more active element as mentioned above, and the ions 373 is configured as an oxidant agent or a catalyze facilitating oxidation reaction in the redox system to include a relative less active element as mentioned above, but not limited thereto. Preferably, and the ions 373 may include sliver ions or sodium ions, and the ion sustainable-released bodies 370 may include silver zeolite or sodium zeolite, but not limited thereto. Accordingly, the anode electrodes 150 may include a zinc electrode, or other suitable metal electrodes for losing electrons.

In this way, the anode electrodes 150 may still perform an oxidation reaction to release electron and the anode ions (such as zinc ions), and the anode ions are then react with the ions 373 (such as silver ions) sustainably released from the ion sustainable-released body 370 (such as silver zeolite) to conduct an ion exchanging process. Through this performance, the anode electrodes 150 and the ions 373 sustainable-released from the ion sustainable-released bodies 370 may also indirectly contact with each other while the ion delivery substance exist, to generate a low level of micro-currents via an ion exchanging process to improve the wound healing process. Due to the highly controlled ion releasing rate, the micro-currents generated in the present embodiment may be precisely controlled at about 0.1 to 30 microamperes (μA), preferably being 1-20 μA, but not limited thereto.

Through these arrangements, the wound dressing 300 according to the second embodiment of the present invention is provided, and which is also allowable to self-generate micro-currents with single electrode technology, for improving the healing process of biological tissues. In the present embodiment, since the electrode layer only includes a single electrode (the anode electrodes 150 for example) and the ion sustainable-released body 370, the landing occupation of the electrodes on the insulating layer 130 is significantly reduced, so as to save cost and improve element efficiency. Furthermore, the ion sustainable-released body 370 is namely the ionized cathode electrode, which is capable to highly control the ion releasing rate of cathode ions, and also to harmonize the distribution thereof, thereby further adjusting the level of the self-generated micro-currents. Accordingly, the ion releasing rate of the cathode ions in the present embodiment is significantly slowdown by disposing the ion sustainable-released body 370, so that, the wound dressing 300 is sufficient to avoid serious biological toxicity caused by excessive ion release. Also, the micro-currents self-generated on the wound dressing 300 may be more durable due to disposing said ionized cathode electrodes. As shown in Table 1 and Table 2 below, the data of a cell cytotoxicity test after 24 hours treatments and after 48 hours treatments, respectively. It is noted that, cells, such as L929 cell line, treated with the wound dressing 300 of the present embodiment show better cell viability after 24 hours treatments and after 48 hours treatments, as in comparison with the same cells (L929 cell line) treated with some polymer materials, such as high-density polyethylene or 10% dimethyl sulfoxide, treated with the wound dressing 100, or treated with a commercial dressing product.

TABLE 1
Cell cytotoxicity test after 24 hours treatments

| Materials | Mean cell viability (%) |
|---|---|
| control (cell only) | 100 |
| high-density polyethylene (HDPE) | 95.53 |
| 10% dimethyl sulfoxide (DMSO) | 0 |
| wound dressing 300 | 88.62 |
| wound dressing 100 | 86.40 |
| other sample (commercial dressing | 3.57 |
| product) | |

TABLE 2
Cell cytotoxicity test after 48 hours treatments

| Material | Mean cell viability (%) |
|---|---|
| control (cell only) | 100 |
| high-density polyethylene (HDPE) | 88.89 |
| 10% dimethyl sulfoxide (DMSO) | 0 |
| wound dressing 300 | 71.56 |
| wound dressing 100 | 39.44 |
| other sample | 0.63 |

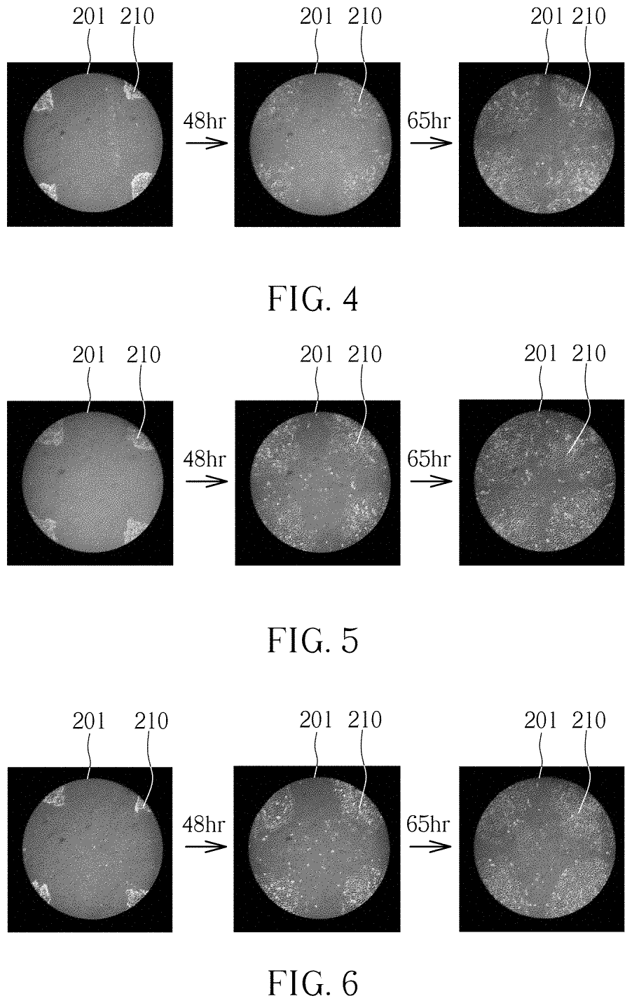

Also, the ion sustainable-released body 370 has a relative smaller size in comparison with metal electrode such as the cathode electrodes 150, and which may be further evenly distributed either disposed on the top surface of the insulating layer 130 or disposed within the entire film of the insulating layer 130. Accordingly, the micro-currents self-generated on the wound dressing 300 of the present embodiment may also be evenly distributed, so as to further improve the therapeutic function of the wound dressing 300. Please refer to Table 3 and FIGS. 4-6, which shown the rate of a cell repairing test after 24 hours treatments. According to the wound closure test, plates 201 full with cells such as A549 cell line are firstly prepared, and a cross line as shown in the left of FIGS. 4-6 is drawn on each of the plates 201 to simulate a tissue wound, and then, various materials as listed in the Table 3 are applied to each plate 201 to record the wound closure after 48 hours and 65 hours. As shown in the Table 3 and the right of FIG. 5, more cells 210 are grown after the treatment of the wound dressing 300, as in comparison with the control cells (as shown in right of FIG. 4) or cells treated with insulating layer 130 only (as shown in the right of FIG. 6). Thus, the wound dressing 300 of the present embodiment provides better wound closure function. Please also understand that although the wound dressing 300 of the present embodiment is exemplified by disposing anode electrode and ionized cathode metal, the wound dressing of the present invention is not limited thereto. In another embodiment, another wound dressing may also be provided by disposing cathode electrode and ionized anode metal under various therapeutic purposes or product requirements.

TABLE 3
Cell repairing test

Record Point

| | 48 hr | 65 hr |
|---|---|---|
| control (no materials) | 24.24% | 58.45% |
| wound dressing 300 | 33.59% | 76.11% |
| other sample (insulating layer 130 only) | 30.54% | 52.88% |

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A wound dressing, comprising:
a substrate;
an insulating layer, disposed on the substrate;
at least one ion sustainable-released body, disposed at the insulating layer, the ion sustainable-released body comprising a plurality of ions; and
at least one electrode, disposed on the insulating layer, wherein the electrode and the ions are functioned as an electron donor and an electron acceptor respectively.

2. The wound dressing accordingly to claim 1, wherein a plurality of the ion sustainable-released bodies is disposed on a top surface of the insulating layer.

3. The wound dressing accordingly to claim 1, wherein a plurality of the ion sustainable-released bodies is disposed within the insulating layer.

4. The wound dressing accordingly to claim 1, wherein a weight ratio between the ion sustainable-released bodies and the insulating layer is 0.01% to 10%.

5. The wound dressing accordingly to claim 1, wherein the ion sustainable-released body comprises:
a carrier; and
the ions covered by the carrier.

6. The wound dressing accordingly to claim 5, wherein a weight ratio between the ions and the carrier is 1% to 5%.

7. The wound dressing accordingly to claim 5, wherein the carrier comprises a plurality of micro-channels for releasing the ions.

8. The wound dressing accordingly to claim 7, wherein a size of each of the micro-channels is about 1 angstrom to 10 nanometers.

9. The wound dressing accordingly to claim 7, wherein the carrier comprises aluminosilicate, mineral, clay or filter.

10. The wound dressing accordingly to claim 1, wherein the ion sustainable-released body comprises silver zeolite or sodium zoelite.

11. The wound dressing accordingly to claim 1, wherein the insulating layer comprises a polymer material.

12. The wound dressing accordingly to claim 11, wherein the polymer material comprises polymer film, rubber, polyurethane material, polyethylene, polyethylene terephthalate, thermoplastic polyurethane, thermoplastic polyester elastomer, or biocompatible resin.
